(19) United States
(12) Patent Application Publication
(10) Pub. No.: US 2023/0255465 A1
(43) Pub. Date: Aug. 17, 2023

(54) Laryngoscope With Video Capture

(71) Applicant: Vandy T. Gaffney, II, Mabelton, GA (US)

(72) Inventor: Vandy T. Gaffney, II, Mabelton, GA (US)

(21) Appl. No.: 17/673,744

(22) Filed: Feb. 16, 2022

(51) Int. Cl. A61B 1/267; A61B 1/05; A61B 1/00; A61B 1/06

(52) U.S. Cl. CPC A61B 1/267; A61B 1/05; A61B 1/00101; A61B 1/00121; A61B 1/0676

(57) ABSTRACT

A light weight, non-bulky, video laryngoscope is provided comprising a handle and a blade. The distal end of the handle has a connector portion for receiving a larynx blade. The larynx blade has an elongated blade portion with an integrated camera, light, and a camera lens wiper. The integrated camera and camera lens wiper are electromechanically connected to the handle via the connector portion. The proximal end of the handle also has a peripheral port for electromechanically connecting to peripheral devices. Within the handle is a battery and electronic components for capturing, storing, and transmitting video and images captured by the integrated camera within the blade. The images can be transmitted via a wired or wireless means to computers, mobile devices, tablets, workstations, and the like. Controls within the handle allow video to be captured, lights to be operated, and condensation to be removed from the camera lens.

### FIELD OF THE INVENTION

The invention relates to an improved laryngoscope with an integrated video camera for wired or wireless video capture.

### BACKGROUND OF THE INVENTION

Laryngoscopy is a medical procedure that allows medical professionals to view and observe the glottis or vocal cords in the larynx of a human. In effect, this procedure is endoscopy of the larynx. Laryngoscopy is also performed to allow for tracheal intubation during general anesthesia; cardiopulmonary resuscitation; surgical procedures; etc.

There are several different types of laryngoscopies including direct, direct fiber-optic, direct video, and indirect. Direct laryngoscopy is an involved procedure wherein the patient lies on their back; while a laryngoscope is inserted into the patient’s mouth to hold down the patient’s tongue; while simultaneously lifting up the patient’s epiglottis. The epiglottis is a flap of cartilage that covers the patient’s larynx, also known as the windpipe. The epiglottis opens during breathing and closes during swallowing. The direct laryngoscopy procedure allows the medical professional to view the patient’s glottis via a direct line of sight. This procedure is most often employed by anesthesiologists and other medical professionals like an otolaryngologist or an anesthetists for endotracheal intubation under general anesthesia. These professionals may also use the laryngoscope in direct diagnostic laryngoscopy with biopsy.

Similar to the direct laryngoscopy procedure described above, fiber-optic laryngoscopy involves placing a small telescope at the end of a flexible fiber-optic cable. The telescope and flexible fiber-optic cable can be inserted through the mouth or nose; and then extended further down the throat to view the glottis. The glottis is then viewable via the telescope and flexible fiber-optic cable as images are transmitted to an attached lens, eye piece, or monitor for visualization. Similarly, direct video laryngoscopy includes an image sensor or a video camera integrated into a flexible cable or blade of the laryngoscope. The video camera captures an image of the glottis and the image is transmitted to an attached monitor allowing view of the larynx.

Indirect laryngoscopy involves the medical professional using a small mirror and light to peer into the patient’s throat. The mirror is on a long handle and is placed against the roof of the patient’s mouth, like a dental mirror. A light is then shined into the patient’s mouth to allow the medical professional to view the reflection of the glottis or larynx in the mirror.

Prior art direct video laryngoscopy (VL) systems have not been very successful in the medical industry. VL systems have been associated with higher rates of complications. VL systems also did not yield higher first-attempt tracheal intubation success rates than direct laryngoscopy system. (see Annals of Intensive Care, “Video laryngoscopy for first-attempt tracheal intubation in the general ward,” by Moon Seong Baek, et. Al.; published Aug. 13, 2018). These systems have failed primarily due to their cost, bulky designs, difficult user interfaces, and their complicated set up. Also, oral secretions, heat and humidity of the patient’s oral cavity and throat often fog up the camera lens of the video laryngoscope, requiring the entire system to be removed and cleaned during use, potentially causing issues with the overall procedure. What is needed in the industry is a VL system that is inexpensive, simple to use, mobile, with an intuitive user interface, non-bulky design, and with systems to prevent or mediate camera fogging.

### SUMMARY OF THE INVENTION

The present invention contemplates a novel video laryngoscopy system that is inexpensive, easy to use, with an intuitive user interface, non-bulky design, easily mobile, and with a fogging and humidity mitigation system for the camera lens.

A first embodiment of the present invention provides a laryngoscope comprising a handle having a distal end, a proximal end, and a middle portion. The distal end of the handle has a connector portion for receiving a larynx blade. Along its distal end, the larynx blade has an integrated camera or image sensor, camera lens, and a fogging and humidity mitigation system for the camera lens. The camera or image sensor can capture images in the optical spectrum, infrared, ultra-violet, or other optical spectrums. The larynx blade further has an elongated portion, followed by a connector portion at its proximal end. The connector portion of the larynx blade allows the integrated camera and camera lens fogging and humidity mitigation system to connect electromechanically, mechanically, electrically, and/or wirelessly to the distal end of the handle via the handle’s connector portion. The proximal end of the handle also has a peripheral port for connecting to peripheral devices. The peripheral port allows the handle to connect electromechanically, mechanically, electrically, and/or wirelessly to a peripheral such as a display screen. The peripheral port can be fixed or adjustable to allow a peripheral device such as a display to be placed in an optimal viewing position. The proximal end of the handle can also contain controls for operating the laryngoscope. Ergonomic grips can be integrated into the middle portion of the handle for holding and controlling the laryngoscope.

In a second embodiment, the distal end of the handle contains a blade connector for electromechanically, mechanically, electrically, and/or wirelessly connecting and engaging a variety of different blade types for the laryngoscope. These different blade types can include Macintosh, Miller, and other types of curved and straight blades. Furthermore, the blades can contain an integrated camera and light source at the distal end of the blade. The camera lens can include a fogging and humidity mitigation system such as an integrated camera lens wiper for removing secretions, condensation, and debris from the camera lens. This fogging and humidity mitigation system can be integrated into the distal end of the blade with the camera lens. Controls for operating the camera and fogging and humidity mitigation systems for removing condensation and secretions from the camera lens can be integrated into the handle at its distal end or integrated into a user interface within a peripheral device. The user interface within the peripheral device can connect with the fogging and humidity mitigation system via the peripheral port in the handle using electromechanical, mechanical, electrical, and/or wireless means.

In still a further embodiment, the proximal end of the handle contains the peripheral port for electromechanically, mechanically, electrically, and/or wirelessly connecting to peripheral devices; and wherein said peripheral devices can include a detachable smart mobile device and display. The peripheral port is powered by electrical and electronic components that are integrated within the handle for capturing, storing, and transmitting video and images captured by the blade’s integrated camera. The connector portion of the larynx blade allows the integrated camera and camera lens fogging and humidity mitigation system to connect electromechanically, mechanically, electrically, and/or wirelessly to the distal end of the handle via the handle’s connector portion and these components further connect to the peripheral port components at the proximal end of the handle. Electromechanical, mechanical, electrical, and/or electronic components for wirelessly transmitting video and images captured by the integrated camera can also be integrated into the handle or into the peripheral device. The handle can contain a battery for powering the electrical and electronic components, the integrated camera, light source, and camera lens wiper.

In a further embodiment, the blade contains a folding mechanism that engages the connector portion of the handle and wherein the folding mechanism allows the blade to be folded 180 degrees relative to the handle portion.

In still a further embodiment, a handle for electrically connecting to a laryngoscope blade at its distal end and a peripheral device at its proximal end. The distal end of the handle having a connector portion for receiving the larynx blade. The larynx blade having an elongated blade portion with a camera sensor and light source. The camera sensor and light source are connected to electrical components within the handle via the connector portion and to the peripheral device.

The above features as well as additional features and aspects of the present invention are disclosed herein and will become apparent from the following description of preferred embodiments of the present invention.

This summary is provided to introduce a selection of aspects and concepts in a simplified form that are further described below in the detailed description. This summary is not intended to identify key features or essential features of the claimed subject matter, nor is it intended to limit the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an exemplary illustration of an embodiment of a laryngoscopy procedure being performed on a patient;

FIG. 2A is an exemplary illustration of an embodiment of the present invention laryngoscope handle and folded blade;

FIG. 2B is an exemplary illustration of a perspective view of an embodiment of the present invention laryngoscope handle and folded blade;

FIG. 3A is an exemplary illustration of an embodiment of the present invention laryngoscope handle and extended blade;

FIG. 3B is an exemplary illustration of an embodiment of the present invention laryngoscope blade with integrated camera and camera lens wiper;

FIG. 4A is an exemplary illustration of an embodiment of the present invention laryngoscope wirelessly connected to an external mobile device;

FIG. 4B is a flow diagram of an embodiment of present invention that illustrates how the laryngoscope wirelessly connects to an external mobile device;

FIG. 5A is an exemplary illustration of an embodiment of the present invention laryngoscope physically connecting with an external peripheral display device;

FIG. 5B is a flow diagram of an embodiment of present invention that illustrates how the laryngoscope physically connects to an external mobile display device;

FIG. 6A is an exemplary illustration of an embodiment of the present invention laryngoscope handle with an integrated blade;

FIG. 6B is an exemplary illustration of an embodiment of the present invention laryngoscope handle with an integrated blade and detachable display;

FIG. 6C is an exemplary illustration of an embodiment of the present invention laryngoscope handle with an integrated blade and wireless display and data transfer; and

FIG. 6D is an exemplary illustration of an embodiment of the present invention laryngoscope handle with an integrated blade and wireless data transfer to a workstation.

### DETAILED DESCRIPTION

The foregoing summary, as well as the following detailed description of illustrative embodiments, is better understood when read in conjunction with the appended drawings. For the purpose of illustrating the embodiments, there is shown in the drawings, exemplary constructions of the embodiments; however, the embodiments are not limited to the specific methods and instrumentalities disclosed. In the drawings:

Before the present device, methods and systems are disclosed and described in greater detail hereinafter, it is to be understood that the devices, methods and systems are not limited to specific devices, methods, specific components, or particular implementations. It is also to be understood that the terminology used herein is to describe particular aspects and embodiments only and is not intended to be limiting.

As used in the specification and the appended claims, the singular forms “a,” “an” and “the” include plural referents unless the context clearly dictates otherwise. Similarly, “optional” or “optionally” means that the subsequently described feature or component may or may not be included, and the description includes instances where the feature or component is included and instances where it is not included.

Throughout the description and claims of this specification, the word “comprise” and variations of the word, such as “comprising” and “comprises,” mean “including but not limited to,” and is not intended to exclude, for example, other components, integers, or steps. “Exemplary” means “an example of” and is not intended to convey an indication of preferred or ideal embodiment. “Such as” is not used in a restrictive sense, but for explanatory purposes.

Disclosed are components that can be used to perform the disclosed device, methods, and systems. These and other specific components are disclosed herein. It is understood, however, that when combinations, subsets, interactions, groups, etc. of these components are disclosed with specific reference to each of the various individual and collective combinations, there can be permutations of these combinations or groups that are not explicitly disclosed. However, each of these groups and combinations of components are specifically contemplated and described herein to arrive at the invention for each device, method, or system. This applies to all aspects of this specification including, but not limited to, combinations of described device components. Thus, if there are a variety of component combinations that can be assembled with the video laryngoscopy system, it is understood that each of the additional component combinations can be used with any of the specific embodiments or combination of embodiments of the disclosed video laryngoscopy system.

As will be appreciated by one skilled in the art, the methods and systems can take the form of an entirely new hardware embodiment, an entirely new software embodiment, or an embodiment combining new software and hardware aspects. References are made herein to the attached drawings. Like reference numerals are used throughout the drawings to depict like or similar elements of the video laryngoscopy system and other components. For the purposes of presenting a brief and clear description of the present invention, the preferred embodiment will be discussed as a system for providing a video laryngoscopy system. The figures are intended for representative purposes only and should not be construed to be limiting in any aspect.

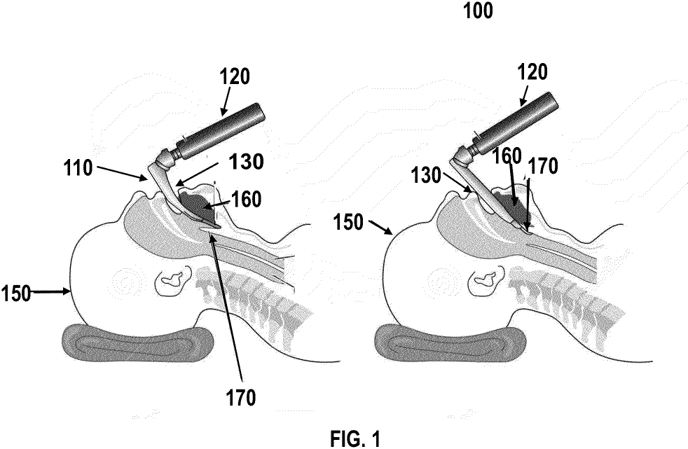

FIG. 1 is an exemplary illustration of a laryngoscopy procedure 100 being performed on a patient 150. Laryngoscopy is an involved procedure 100 wherein the patient 150 lies on their back while a laryngoscope 120 and blade 110 is inserted into the oral cavity 130 (the mouth) to hold down the patient’s tongue 160 while also lifting up the epiglottis 170. The epiglottis 170 is the flap of cartilage that covers the windpipe. The epiglottis 170 opens during breathing and closes during swallowing. When the blade 110 is inserted into the oral cavity 130, the medical professional is able to manipulate the laryngoscope 120 so that the glottis can be viewed via direct line of sight. This procedure is most often employed by anesthetists for endotracheal intubation under general anesthesia, but also in direct diagnostic laryngoscopy with biopsy.

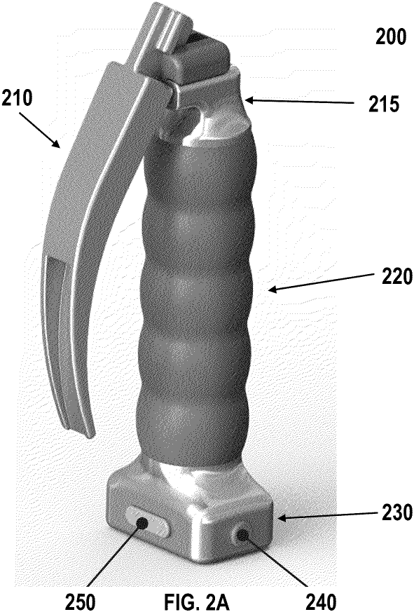

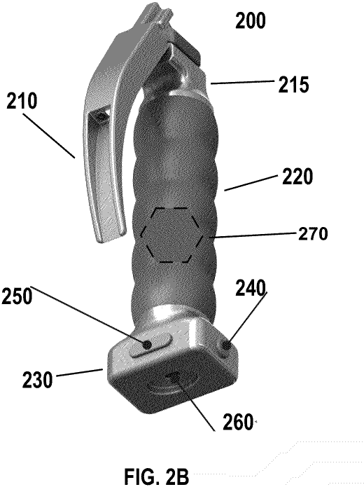

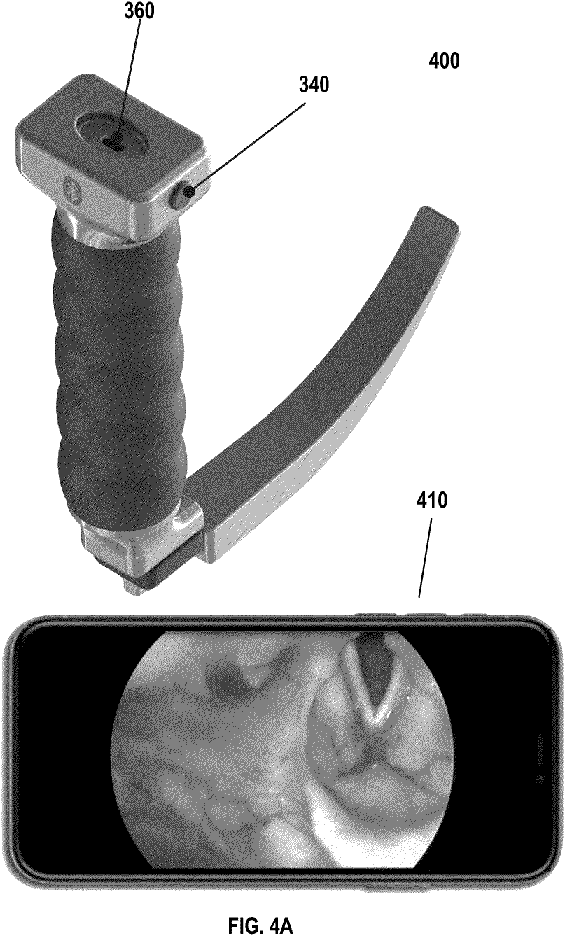

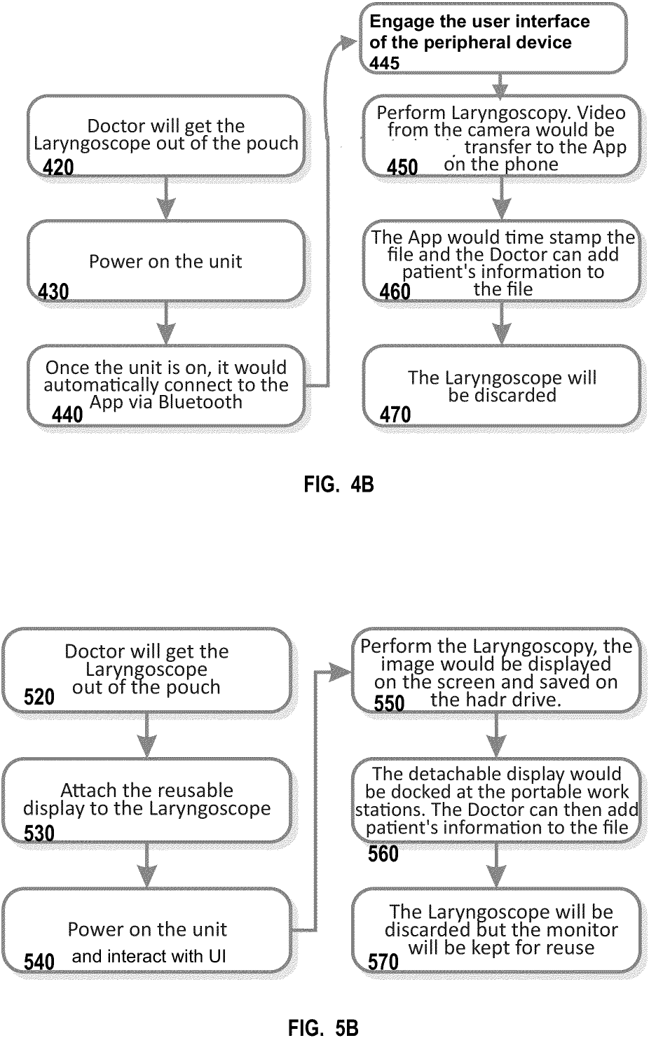

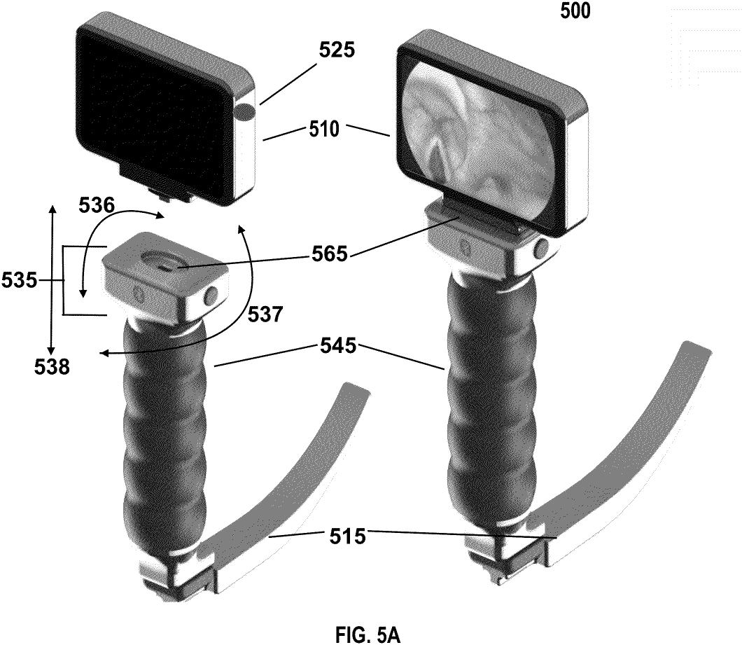

FIG. 2A is an exemplary illustration of an improved laryngoscope 200 of the present invention. The laryngoscope 200 comprises a handle 220 portion that rests on a base 230 at a proximal end. At the distal end, a connector 215 is found. The connector 215 connects the blade 210 with the laryngoscope 200 handle 220. The blade 210 can consist of Macintosh, Miller, and other types of curved and straight blades. The blade 210 can feature other shapes and contours to allow comfortable insertion. The blade 210 can be custom fitted to the patient. The blade 210 can include a folding mechanism that engages the connector 215 portion of the handle 220 and wherein the folding mechanism allows the blade 210 to be folded 180 degrees relative to the handle 220 portion. In a further embodiment, the blade 210 can be fixedly attached to the handle 220. Further along the proximal end of the handle 220 are control buttons 250 for operation of the laryngoscope 200 and a button 240 for controlling the fogging and humidity mitigation system. In a further embodiment, these control buttons 250 and 240 can be integrated into a user interface 445 in the peripheral device 410, 510 as shown in FIGS. 4 and 5.

FIG. 2B is an exemplary illustration of an improved laryngoscope 200 of the present invention. The laryngoscope 200 comprises a handle 220 portion that rests on a base 230 at a proximal end. Further at the proximal end is a peripheral connector 260. The peripheral connector 260 can be fixedly attached to the handle 220 or flexibly attached to the handle 220. This peripheral connector 260 can be Universal Serial Bus (USB) connector, IEEE 1394 (FireWire), or any other wired data transfer and power connector. Within the handle 220 additional electronics can be placed to allow the peripheral connector 260 to attached to a peripheral 410, 510 electromechanically, mechanically, electrically, and/or wirelessly. An electronics package 270 within the handle 220 can include wired and wireless data transfer components, wireless fidelity modems (WIFI), Bluetooth modems (Bluetooth), near field communications (NFC) technologies, ultra-wide band (UWB), mobile Cellular, Satellite, and the like. This allows the device to be used with tele-health services, to store captured video and data in the cloud, and the like. The electronics package 270 can also contain data storage components, microprocessors, global positioning technologies, gyroscopes, accelerometers, and the like. All of these storage and wireless transmission components can include end-to-end encryption technologies for privacy, data security, and compliance with the United States of America Health Insurance Portability and Accountability Act (HIPAA) and the European Union’s General Data Protection Regulation (GDPR). The use of cellular, satellite, and other wireless transmission protocol server to reduce latency and allow retain remote video capture of the laryngoscope 200 in operation. The electronics package can be controlled via buttons 250 for power and image capture as well as button 240 for controlling the fogging and humidity mitigation system. In a further embodiment, these control buttons 250 and 240 can be integrated into a user interface 445 in the peripheral device 410, 510 as shown in FIGS. 4 and 5.

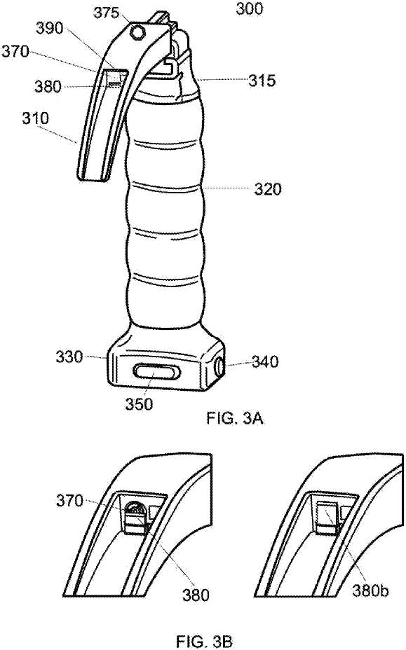

FIG. 3A provides an exemplary embodiment of the laryngoscope 300 of the present invention. In FIG. 3, the blade 310 contains a high-resolution, water proof, camera sensor 370 (e.g. a video camera, infra-red camera, or other image sensor) wherein the camera sensor 370 has an exposed camera lens 375. The blade 310 also contains a (camera) lens wiper 380 and a light source 390. The lens wiper 380 comprises the bulk of the fogging and humidity mitigation system, by providing the lens wiper 380 to remove fog, moisture, and debris from the camera lens 375. For brevity, throughout the remainder of this disclosure, the camera lens 375 and camera sensor 370 are referred to as one unit, the camera sensor 370. When the blade 310 is inserted into the patients 150 mouth as shown in FIG. 1, the camera sensor 370 captures images for evaluation. In an embodiment, the camera sensor 370, lens wiper 380, and light source 390 are integrated into the blade 310 such that, the blade 310 and all its components are detached from the handle 320 and disposed of after use. In a further embodiment, the camera sensor 370 and light source 390 are separate components that are integrated into the blade 310 when the blade 310 is attached to the handle 320. The camera sensor 370 and the light source 390 and can be shielded by separate lens within the blade and/or protective sleaves over the electronic components, such they are not exposed to the patient. This allows the blade 310 can be disposed of after use and the camera sensor 370 and light source 390 can be reused.

Further in FIG. 3A, in an embodiment, buttons 340, 350 are mounted along the base 330. However, these buttons can be mounted on the handle 320, the connector 315, or other portions of the laryngoscope 300. In a further embodiment, these control buttons 250 and 240 can be integrated into a user interface (UI) 445 in the peripheral device 410, 510 as shown in FIGS. 4A through 5B. Button 350 allows for power on and off; video start and stop; and/or for recording start and stop. Button 340 allows any condensation and debris to wiped from the camera lens 375 using the lens wiper 380 or other fogging and humidity mitigation system. To further assist with aiding visual imagery of the glottis 170, a light source 390 is also integrated into the blade 310. In an embodiment, the light source 390 can be a light emitting diode (LED); organic light emitting diode (OLED); incandescent; fluorescent; and the like. This light source 390 can also be controlled by the button 350 or via a user interface 445. The blade 310 electromechanically, mechanically, electrically, and/or wirelessly connects with the handle 320. This allows the blade 310 and its camera sensors 370, lens wiper 380, and light source 390 to be powered and controlled by buttons 340 and 350 in the base 330 of the handle 320. In an embodiment, the blade 310 contains its own power source and electronics 375 for controlling the camera sensors 370, storing images, and transmitting images while still connecting with the handle 320.

FIG. 3B, further illustrates the fogging and humidity mitigation system of camera sensor 370 and lens wiper 380. When button 340 is pressed or selected in the user interface 445, the lens wiper 380 is engaged to wipe and clean the camera lens 375. Further, the lens wiper 380b completely engages the camera lens 375 to clean it. After the camera lens 375 has been cleaned, the lens wiper 380 completely returns to its stored position. For brevity, throughout the remainder of this disclosure, the camera lens 375 and camera sensor 370 are referred to as one unit, the camera sensor 370. The lens wiper 380 can comprise a felt coded plastic material, fabric coded plastic material, rubber sponge materials, and the like.

FIG. 4A illustrates and exemplary embodiment of the invention wherein external peripheral devices 410 such as mobile smart phones, tablets, laptops, and other computing devices can connect to the laryngoscope 400. The connections can occur via a peripheral connector 360 for wired connections including USB, FireWire, or other wired connections. The peripheral connector 360 can also be a wireless transmitter. This would allow the laryngoscope 400 to also connect wirelessly via short-range wireless standard (i.e. Bluetooth), wireless fidelity (WiFi), near field communications (NFC), ultra-wide band (UWB), or other wireless means to the external peripheral devices 410. Within the peripheral device 410, an application executing a user interface (UI) can be used to control the camera sensor 370, lens wiper 380, and light source 390, and other aspects of the laryngoscope 400. Video from the camera sensor 370 can be supplied directly to the peripheral device 410 for image capture, applying metadata, storage, and transmission. The data captured by the camera sensor 370 and processed by the peripheral device 410 can also be transmitted and stored via electronics 270 within the handle 320 of the laryngoscope 300, 400; within the peripheral device 410; or both for later upload and transmission.

Turning now to FIG. 4B, a system for pairing the laryngoscope 400 to a peripheral 410 is outlined. The device is simply removed from its pouch 420 and powered on 430 by for example pressing a power button 350 on the handle, on the peripheral device 410, or via a UI executing on the peripheral device 410. Once the laryngoscope 400 is powered on, it can manually or automatically pair 440 with a peripheral device 410 that is running an integrated UI application. This automatic pairing 440 can occur via Bluetooth, WIFI, NFC, UWB, or other wireless standards. The blade 210, 310 of the laryngoscope 400 is attached to the handle 220, 320 wherein a provider can manipulate the handle 220, 320 and the blade 210, 310 to perform a laryngoscopy 100. Video from the integrated camera sensor 370 within the blade 210, 310 is transferred 450 via electronics in blade 210, 310 and/or the handle 220, 320 to the peripheral device 410. This video transfer 450 between the integrated camera sensor 370 and the peripheral device 410 can occur via wired or wireless means. At step 460, the application executing the peripheral device 410 can be used to add metadata, medical notes, and other information to the video being captured by the laryngoscope 400. Once the procedure is completed, the peripheral device 410 is disconnected and the entire laryngoscope 400 and blade 310 can be discarded at step 470.

FIG. 5A provides an alternative embodiment of the laryngoscope 500. Here, a dedicated external peripheral display device 510 is provided. The peripheral display device 510 may be a wireless device or an integrated device. In an exemplary embodiment, the peripheral display device 510 is a compact detachable display for receiving video from the camera sensor 370. The peripheral display device 510 contains electronic computing components 525 for processing, storing, and transmitting the imagery captured from the blade’s 310, 515 camera sensor 370. The peripheral display device 510 is further capable of executing computing operations using its electronic computing components 525. In an embodiment, the peripheral display device 510 executes a user interface (UI) that provides full control over the laryngoscope 500, such that an operator can control video capture from the camera sensor 370, the lens wiper 380, and the light source 390 as illustrated in FIG. 3A. The peripheral display device 510 can be attached directly to the laryngoscope 500 handle 545 via the peripheral port 565. Once the laryngoscope handle 545 and blade 310, 515 have been used on a patient, they can be discarded by detaching the peripheral display device 510 so that it can be used on the next laryngoscope 500 device.

In FIG. 5A the laryngoscope 500 comprises a handle 545 portion that rests on a base 535 at a proximal end. The base 535 can be fixedly attached to the handle 545 or flexibly attached to the handle 545. In an embodiment, the base 535 is flexibly attached to the handle 545 in a manner that allows the base to tilt forward and back 536 and left to right 537 relative to the handle 545. In a further embodiment, the base 535 can extend up and down 538 relative to the base 545. In an embodiment, the base 535 extend up and down 538 telescopically relative to the base 545. Further at the proximal end of the base 535 is a peripheral connector 565. The peripheral connector 565 rest within the base 535. This peripheral connector 565 can be Universal Serial Bus (USB) connector, IEEE 1394 (FireWire), or any other wired data transfer and/or power connector. Within the handle 545 additional mechanical components and electronics can be placed to allow the peripheral connector 565 to attached to the peripheral display device 510 electromechanically, mechanically, electrically, and/or wirelessly.

Turning now to FIG. 5B, a system for pairing the laryngoscope 300, 400, 500 to the peripheral display device 510 or another external device is outlined. The laryngoscope 500 is simply removed from its pouch 520 and the peripheral display device 510 is attached 530 to the disposable laryngoscope 300, 400, 500. The combined unit is then powered on 540 by pressing the power button 350 and/or interacting with the user interface 445. Once the laryngoscope 500 is powered on, the laryngoscopy 100 is performed 550. Video from the camera sensor 370 within the blade 310 is transferred 550 via wired or wireless electronics within the blade 310 and handle 320 to the peripheral display device 510. The peripheral display device 510 can have integrated storage for retaining the video. At step 560, the peripheral display device 510 is removed and docked at a workstation wherein an application executing on a workstation can add metadata and other information to the video that was captured by the camera sensor 370. Once the procedure is completed, the entire laryngoscope 300, 400, 500 and blade 310 is discarded at step 570; whilst the peripheral display device 510 is retained for later use.

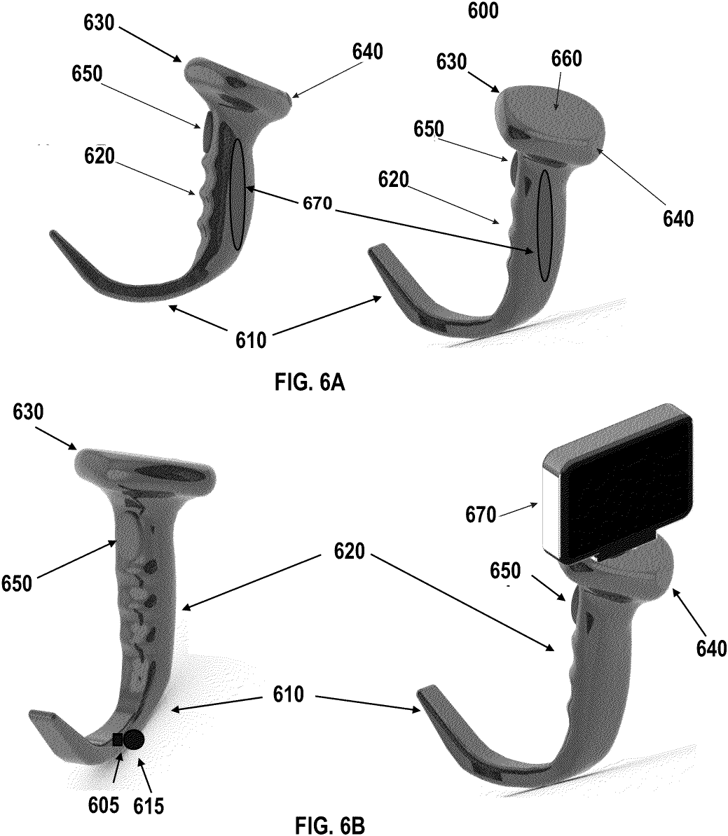

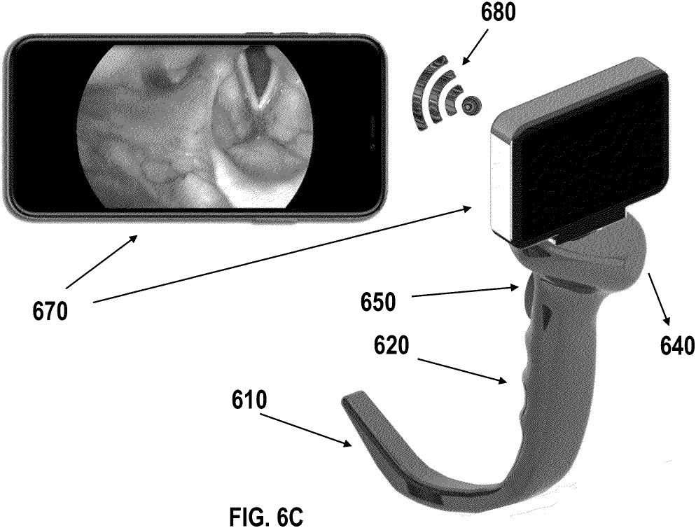

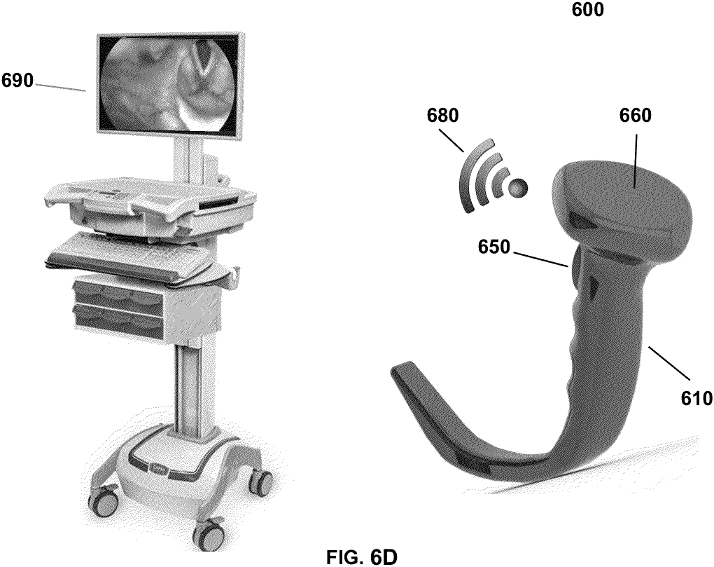

Further in FIGS. 6A - 6C is an exemplary illustration of an embodiment of an improved laryngoscope 600 of the present invention. Staring with FIG. 6A, the laryngoscope 600 comprises a substantial handle 620 portion that connects to a base 630 at a proximal end and a blade portion 610 at a distal end. Further at the proximal end is a peripheral connector 660. This peripheral connector 660 can be Universal Serial Bus (USB) connector, IEEE 1394 (FireWire), or any other wired or wireless data transfer and power connector. Additional electronics and controls 670 can be integrated within the handle 620. For example, a battery for powering the camera sensor 370, 605; lens wiper 380,615; light source 390, and other electronics can be stored within the handle 620. The electronics package 670 within the handle 620 can include wired and wireless data transfer components (WIFI, Bluetooth, NFC, UWB, etc.). The electronics package 670 can also contain data storage components, microprocessors, global positioning technologies, gyroscopes, accelerometers, and the like. Operation of features of the electronics package can be controlled via the UI 445 or via buttons 650 for power and image capture, as well as button 640 which operates the lens wiper. For brevity we describe the controls as buttons 650, 640 however the controls could also be touch controllers, user interface buttons 445, touch displays, leavers, switches, dials, rotary dials, and the like. Furthermore, the functionality of buttons 650 and 640 can vary. For example, button 640 could be used for power and image capture, while button 650 could operate the lens wiper 380, 615. In a further embodiment, the blade 610 incorporates a camera sensor 370, 605; lens wiper 380, 615; and light source 390 as described in the discussion of FIGS. 3A - 4A above. The camera sensor 370, 605 is triggered by control button 650 or via a UI 445 executing within a peripheral device 670. The camera sensor 370, 605 can be cleaned by the lens wiper 380, 615 by pressing button 640. The functions of control buttons 640 and 650 can also be integrated into the UI 445 for wired or wireless operation.

Further FIG. 6B provides an alternative embodiment of the laryngoscope 600 system. Like the system in 5A, here, a dedicated external peripheral display device 670 is provided. The peripheral display device 670 may be an external device or an integrated device. In an exemplary embodiment, the peripheral display device 670 is a compact, detachable computing device with a display for receiving video and data from the camera sensor 370, 605 and for storing, processing, and transmitting the imagery captured in the video. The peripheral display device 670 could also be a mobile device, smart phone, tablet computer, wireless high-definition multimedia interface (HDMI) wired or wireless video capture device, and the like. The peripheral display device 670 can be attached directly to the laryngoscope 600 via the peripheral port 660 or it can connect wirelessly to device 640. Once the laryngoscope 600 has been used and is ready to be discarded, the peripheral display device 670 can be detached and used on the next laryngoscope 600 device.

FIG. 6C provides a system for pairing the laryngoscope 600 to the peripheral display device 670 and/or other external devices. The peripheral display device 670 is attached to the disposable laryngoscope 600. The peripheral display device 670 can connect to the laryngoscope 600 via wired or wireless connection. The combined unit is then powered on by pressing the power button 650 or via the user interface 445. Once the laryngoscope 600 is powered on, the laryngoscopy 100 can be performed. Data and Video from the integrated camera sensor 370, 605 within the blade 610 is transferred via wired or wireless electronics in the handle 620 to the peripheral display device 670. The peripheral display device 670 is a computing device with integrated storage for retaining the video. The peripheral display device 670 can also be an external mobile device, mobile phone, tablet computer, or laptop as described above. In an embodiment the external display device 670 functions separately from the laryngoscope 600, such that, video and data is transferred wirelessly 680 using a short-range wireless technology (e.g. Bluetooth), wireless fidelity (WIFI), near field communications (NFC), ultra-wide band (UWB), and/or other wireless protocols. Once the procedure is completed, the entire laryngoscope 600 and blade 610 is discarded but the peripheral display device 670 is retained for later use.

FIG. 6D illustrates a further embodiment of a laryngoscope 600 and blade 610 that is used without an attached display. Instead, in an embodiment, the laryngoscope 600 is used to perform a laryngoscopy procedure while the blade wirelessly 680 transmits data and video to a workstation 690. The wireless 680 data and video transfer can occur in a real time and/or it can be uploaded to the workstation 690 later. In a further embodiment, the data and video transfer can occur via a wired or wireless connection between the peripheral port 660 and the workstation 690. The capture and transfer of video can be controlled by control input 650 or via a user interface 445. The workstation 690 may also include video monitors, computer workstations, and other video and data capture devices.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A laryngoscope comprising:
a handle for connecting to a laryngoscope blade at its distal end with integrated components for connecting to a peripheral device via a port at its proximal end;
said distal end of the handle having a connector portion for receiving the larynx blade;
said larynx blade having an elongated blade portion with an integrated camera, light source, and a camera lens wiper;
wherein said integrated camera, light source, and camera lens wiper are connected to components within the handle via the connector portion; and to said peripheral device.

2. The laryngoscope according to claim 1, wherein the proximal end of the handle contains controls for operating the laryngoscope.

3. The laryngoscope according to claim 2, wherein the controls for controlling the laryngoscope are found within a user interface executing on a peripheral device.

4. The laryngoscope according to claim 1, wherein the distal end of the handle contains a blade connector for electromechanically, electrically, mechanically, or wirelessly connecting and engaging a variety of blade types for the laryngoscope.

5. The laryngoscope according to claim 4, wherein the blade types include Macintosh, Miller, and all other types of curved and straight blades.

6. The laryngoscope according to claim 5, wherein the blades contain an integrated camera and light at distal end of the blade and wherein the blade electromechanically, electrically, and/or mechanically engages the connector portion of the handle.

7. The laryngoscope according to claim 6, wherein the camera lens has a camera lens wiper for removing condensation and debris from the camera lens.

8. The laryngoscope according to claim 7, wherein the controls for operating the wiper mechanism for removing condensation and debris from the camera lens is mounted on the proximal end of the handle.

9. The laryngoscope according to claim 2, wherein the proximal end of the handle contains the peripheral port for electromechanically, electrically, mechanically, or wirelessly connecting to peripheral devices; and wherein said peripheral device is a detachable smart mobile device or display.

10. The laryngoscope according to claim 2, wherein the handle contains electrical and electronic components for capturing, storing, and transmitting video and images captured by the integrated camera.

11. The laryngoscope according to claim 10, wherein the handle contains electrical and electronic components for wirelessly transmitting video and images captured by the integrated camera.

12. The laryngoscope according to claim 11, wherein the handle contains a battery along with electrical and electronic components for powering the integrated camera, light, camera lens wiper, and electronic components.

13. The laryngoscope according to claim 2, wherein the blade contains a folding mechanism that engages the connector portion of the handle and wherein the folding mechanism allows the blade to be folded 180 degrees relative to the handle portion.

14. A video laryngoscope, the system comprising:
a handle having a connector portion for receiving a larynx blade at its distal end;
said larynx blade having an elongated blade portion with an integrated camera and a lens wiper;
said handle containing a battery and electronics for powering, operating, capturing, storing, and transmitting video and images from the integrated camera; and
wherein said handle has a peripheral port at its proximal end for connecting to peripheral devices.

15. The video laryngoscope according to claim 14, wherein the distal end of the handle contains a blade connector for electromechanically, electrically, or mechanically connecting and engaging a variety of blade types for the laryngoscope.

16. The video laryngoscope according to claim 15, wherein the blade types include Macintosh, Miller, and all other types of curved and straight blades.

17. The video laryngoscope according to claim 16, wherein the blades contain an integrated and camera and light at its distal end.

18. The video laryngoscope according to claim 17, wherein the camera lens has a lens wiper for removing condensation and debris from the camera lens.

19. The video laryngoscope according to claim 17, wherein the blade contains a folding mechanism that engages the connector portion of the handle and wherein the folding mechanism allows the blade to be folded 180 degrees relative to the handle portion.

20. The video laryngoscope according to claim 17, wherein the handle wirelessly transmits video and images from the integrated camera to a peripheral display and/or storage device.
